(19) United States
(12) Patent Application Publication
(10) Pub. No.: US 2025/0333706 A1
(43) Pub. Date: Oct. 30, 2025

(54) METHOD OF INCREASING STEM CELL PRODUCTION

(71) Applicant: The Crowley Center for Rgenerative Biotherapeutics, LLC, Rochester, NY (US)

(72) Inventors: Thomas James Conley, Fairport, NY (US); Frank Razavi, Fairport, NY (US); Michael Crowley, Rochester, NY (US); Erin Crowley, Rochester, NY (US)

(21) Appl. No.: 18/860,812

(22) PCT Filed: Apr. 26, 2023
(86) PCT No.: PCT/US2023/019968

§ 371 Date: Oct. 28, 2024

Related U.S. Application Data
(60) Provisional application No. 63/334,849, filed on Apr. 26, 2022.
(60) Provisional application No. 63/343,755, filed on May 19, 2022.

(51) Int. Cl. C12N 5/0775; A61K 35/28

(52) U.S. Cl. CPC C12N 5/0665; A61K 35/28; C12N 2533/90

(57) ABSTRACT

A method of growing stem cells for therapeutic administration in a subject in need thereof is disclosed herein. The method includes separating stem cells and a protein mix from Wharton's Jelly matrix, separating the stem cells from the protein mix, and depleting bulk proteins from the protein mix to generate a depleted protein mix. The method further includes enriching the depleted protein mix to generate an enriched protein mix, and combining stem cells with an enriched protein mix to increase stem cell yields by a factor of as compared to traditional methods.

### CROSS REFERENCES TO RELATED APPLICATIONS

The following application claims priority under 35 U.S.C. § 119 (e) to U.S. Provisional Patent Application Ser. No. 63/343,755 filed May 19, 2022 entitled METHOD OF INCREASING STEM CELL PRODUCTION and claims priority under 35 U.S.C. § 119 (e) to co-pending U.S. Provisional Patent Application Ser. No. 63/334,849 filed Apr. 26, 2022 entitled METHOD OF INCREASING STEM CELL PRODUCTION. The above-identified applications are incorporated herein by reference in their entireties for all purposes.

### TECHNICAL FIELD

The present disclosure generally relates to a method of increasing stem cell production, and more particularly to a method of increasing stem cell production that is xeno-free and serum free and generates stem cells for use in treatment of humans.

### BACKGROUND

Stem cell acquisition, particularly Mesenchymal Stem Cell (MSC) acquisition is time consuming, inefficient, and expensive. Currently, MSC treatments are limited by a cost of production of the MSCs. The FDA will not approve use of MSCs in humans that have been produced using animal proteins and/or serums. Production of MSCs under such conditions reduces yield, and renders the cost of developing treatments using MSCs too high for pharmaceutical companies, insurance companies, and hospitals.

Exosomes are small, membrane-bound vesicles that are released by cells into the extracellular space and which contain a wide range of biomolecules including deoxyribonucleic acid (DNA), ribonucleic acid (RNA), proteins, and lipids. Exosomes play an important role in intercellular communication and transport of various molecules to facilitate cell-to-cell communication. Exosomes have been shown to be involved in a wide range of physiological processes, including cell proliferation, differentiation and apoptosis. Exosome therapy has a wide range of potential applications in the world of medicine, and beyond. Exosome are usable for the delivery of therapeutic agents, including drugs, siRNA and other active ingredients directly to cells, which is thought to be more effective than traditional methods of applying drugs. Exosomes protect cells from damage and may have future applications in treating a wide range of conditions including cancer, Alzheimer's disease, diabetes, and others.

Similarly, exosomes have applications in cosmetics industry as they can deliver active ingredients directly to skin cells. Generating a substantial supply of exomes cost effectively presents significant problems.

### SUMMARY

One aspect of the present disclosure comprises a method of growing stem cells for therapeutic administration in a subject in need thereof. The method includes separating stem cells and a protein mix from Wharton's Jelly matrix, separating the stem cells from the protein mix, and depleting bulk proteins from the protein mix to generate a depleted protein mix. The method further includes enriching the depleted protein mix to generate an enriched protein mix, and combining stem cells with an enriched protein mix.

Another aspect of the present disclosure comprises a method of deriving extracellular vesicles from stem cells for administration in a subject in need thereof. The method includes separating stem cells and a protein mix from Wharton's Jelly matrix, separating the stem cells from the protein mix, and depleting bulk proteins from the protein mix to generate a depleted protein mix. The method further includes enriching the depleted protein mix to generate an enriched protein mix, combining stem cells with an enriched protein mix, incubating the stem cells with the enriched protein mix to generate stem cell enriched media, and harvesting the extracellular vesicles generated therefrom.

Yet another aspect of the present disclosure comprises a method for preparing a population of mesenchymal stem cells (MSC) for therapeutic administration to a subject in need thereof, the method comprises separating stem cells from a protein mix, depleting bulk proteins from the protein mix to generate a depleted protein mix, enriching the depleted protein mix to generate an enriched protein mix, concentrating stem cells, combining the stem cells with the enriched protein mix, incubating the stem cells with the enriched protein mix to generate stem cell enriched media, and, harvesting MSCs generated therefrom.

### BRIEF DESCRIPTION OF THE DRAWINGS

The foregoing and other features and advantages of the present disclosure will become apparent to one skilled in the art to which the present disclosure relates upon consideration of the following description of the disclosure with reference to the accompanying drawings, wherein like reference numerals, unless otherwise described refer to like parts throughout the drawings and in which:

FIG. 1 illustrates a flow diagram for a method of isolating and growing mesenchymal stem cells (MSC) and vesicles in accordance with one example embodiment of the present disclosure;

FIG. 2 illustrates a yield of a method of isolating and growing mesenchymal stem cells (MSC) compared to a traditional method of isolation and growth, in accordance with one example embodiment of the present disclosure;

FIG. 3 illustrates growth of the mesenchymal stem cells (MSC) after seven days utilizing prior art growth process; and

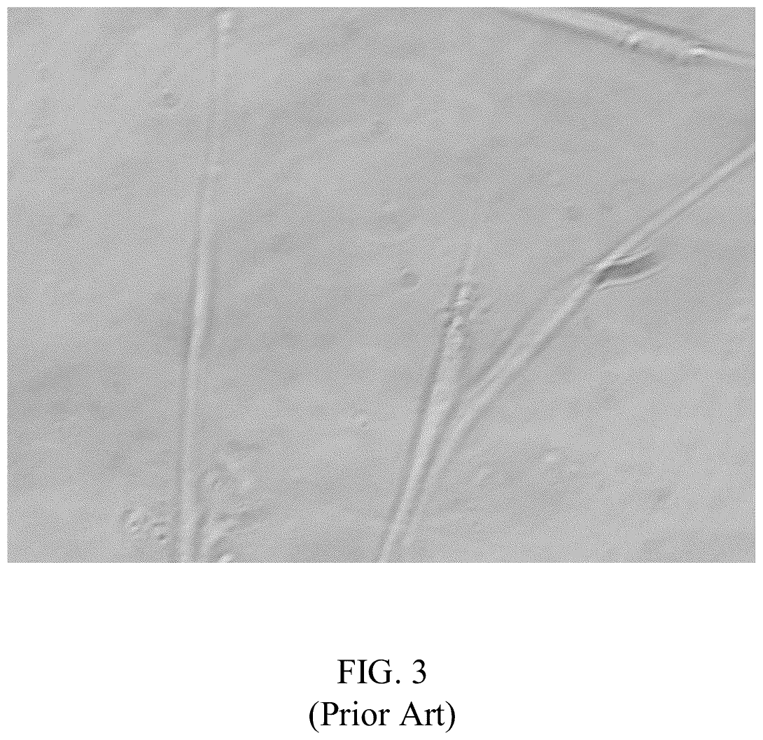

FIG. 4 illustrates growth of the mesenchymal stem cells (MSC) after seven days utilizing the method of FIG. 1, in accordance with one example embodiment of the present disclosure.

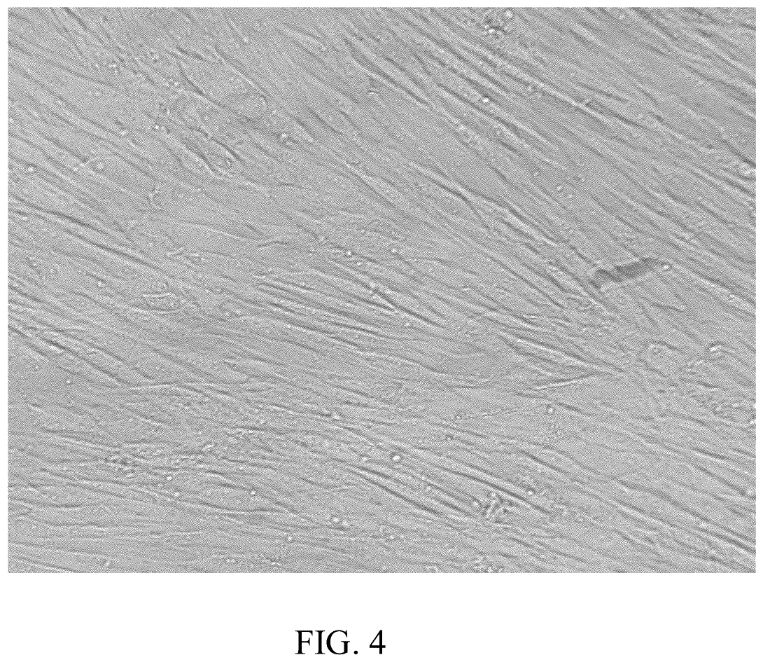

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

Referring now to the figures generally wherein like numbered features shown therein refer to like elements throughout unless otherwise noted. The present disclosure generally relates to a method of increasing stem cell production, and more particularly to a method of increasing stem cell production that is xeno-free and serum free and generates stem cells for use in treatment of humans.

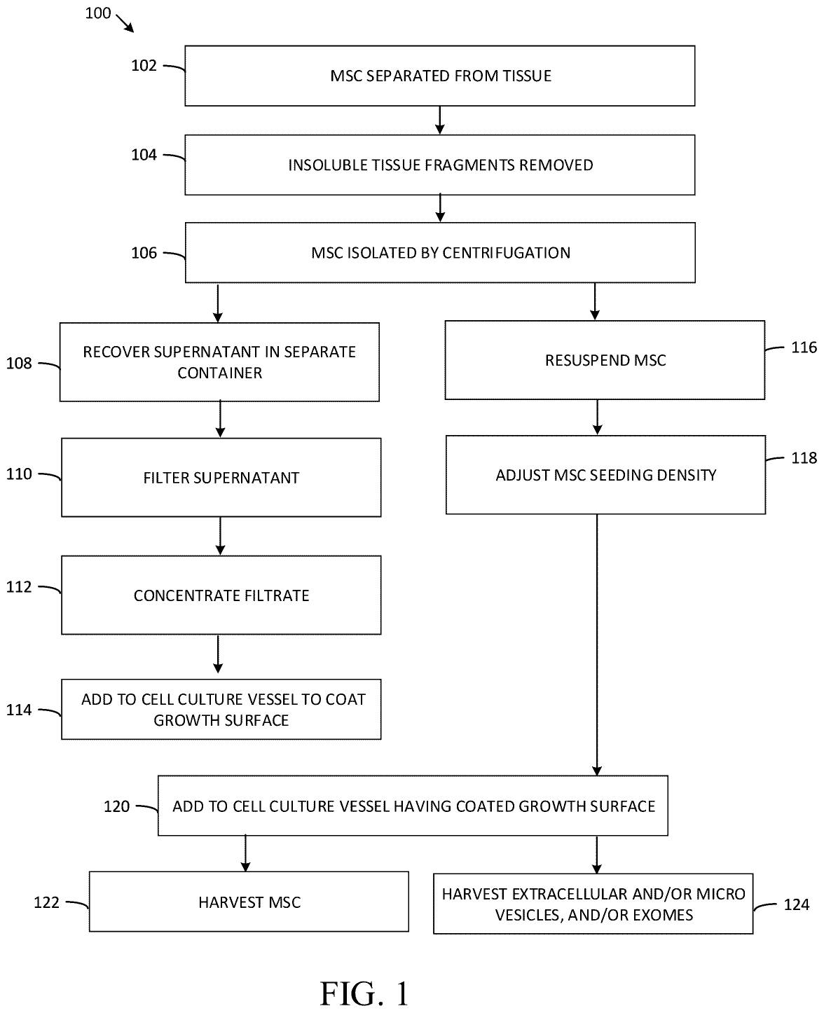

FIG. 1 illustrates a method 100 for improving primary acquisition of umbilical cord mesenchymal stem cells (MSC). Prior to growth, MCSs are separated and cultured. In this example embodiment, the MSCs are separated and cultured by plate laying, fluid infusion, fluid replacement and the like, wherein the temperature is between 35° C. to about 40° C. In one example embodiment, the temperature for the steps of separating and culturing the MSCs is 37° C. In one example embodiment, the method 100 shortens the culture time of the primary MSC (e.g., from 7-14 days to 3-10 days), stably carries out multiple rounds of primary MSC culture, is simple and convenient, and has strong practicability. In one example embodiment, the cells are harvested at or before passage 5, and can accommodate passaging cells through passage 12. Passaging is the procedure of harvesting cells from a culture, transferring the cells to one or more culture vessels with fresh growth medium, and using those cells to start new cultures. It is also referred to as subculturing. Stated another way, the method 100 has wide application prospects for primary MSC culture, application, and the like.

An additional step of method 100, a culture method for isolating MSCs at considerably higher yields, as compared with traditional methods of culture, while maintaining 95-99% purity. Utilizing steps within method 100, during or after cell expansion allows for collection and concentration of extracellular vesicles produced by MSCs and establishes related quality control and evaluation experiments. In one example embodiment, the MSC secretion is applied to the industries of cosmetics, health care products and the like. Additionally, non-human MSC isolation and expansion is completed with the method 100.

At 102 of the method 100, the MSCs are separated from the tissue of an umbilical cord. In one example embodiment, the MSCs are separated by mechanical dissociation. In another example embodiment, upon receipt of the umbilical cord and removal of it from transport solution, the umbilical cord is dissected to isolate a Wharton's Jelly matrix from other umbilical cord components (e.g., mechanical separation). In this example embodiment, upon receipt of umbilical cord, the date and approximate time are recorded. In one example embedment, an outer package housing the umbilical cord is opened, and pathogen screening results are located and reviewed. Responsive to the umbilical cord clearing a pathogen screen, a Regenerelle lot number on corresponding test results is assigned. The umbilical cord is then transported to a laboratory. Exterior surfaces of primary specimen container are sterilized and places into cleanroom transfer window. Following a successful cleanroom pressure check and gowning procedure, the umbilical cord is retrieved from a transfer window and disinfected prior to placement into bio safety cabinet (BSC). The umbilical cord is transferred to a culture dish (e.g., a new 100 mm×20 mm culture dish). It is understood that in some example embodiments multiple umbilical cords can be processed at once. In one example embodiment, a scalpel is used to cut the umbilical cord from a placenta (if present) and divide the umbilical cord into two equal sections. The two sections are placed in a sterile container filled with sterile MSC media (e.g., a serum free, xeno free media).

In one example, part of separating the MSC from the tissue includes, wherein responsive to being isolated from the umbilical cord, the Wharton's jelly is cut into fragments (e.g., roughly 5 mm³ in size). In another example embodiment, part of separating the MSC from the tissue also includes, wherein a plurality of fragments (e.g., 15-20 fragments) having a threshold volume (e.g., 1-2 mL) are transferred to dissociators with media. One such example dissociator is the C-tube dissociators sold by Miltenyi. In one example embodiment, the dissociator has a dissociator volume of media (e.g., 8 mL of MSC media). The dissociators are placed onto an instrument (e.g., a gentleMACs instrument) and tissue is dissociated into a single cell suspension. In one example embodiment, the instrument mechanically dissociates the plurality of fragments into a single cell suspension.

Following the removal of the insoluble tissue fragments (e.g., such as by the mechanical dissociation of plurality of fragments from the Whartons Jelly tissue), at 104, insoluble tissue fragments from the separated MSCs are removed. In this example embodiment, the single cell suspensions, generated from the separating the MSCs from the tissue, and remaining tissue are passed through a cell strainer. One such example cell strainer is the PluriSelect cell strainer.

At 106, the MSCs are isolated by centrifugation. In one example embodiment, MSCs are collected by centrifugation at a cell collection rate (e.g., 400 xg for minutes) to form a cell pellet.

Following centrifugation, at 108, a resulting supernatant is recovered in a separate container. At 110, the resulting supernatant from the separate container is filtered. In one example embodiment, the resulting supernatant from the separate container is filtered through a sterilizing filter (e.g., a 0.22 μm sterilizing filter) into a receiver bottle. In one example embodiment, the resulting supernatant or protein mix is transferred into the upper chamber of a centrifugal filter device, having a first membrane for sterilization and/or filtering. One such example centrifugal filter device is a Pall JumboSep centrifugal filter device containing insert number OD300C65. In one example embodiment, the centrifugal filter device is an ultrafiltration device having a molecular weight cut off (MWCO) of 300 kDa.

In this example embodiment, one or more centrifugal filter devices containing the resulting supernatant from the separate container are centrifuged in a centrifuge. In this example embodiment, the high molecular weight proteins are retained by the filter device as retentate, and filtrate, that has gone through the filter device, is collected. One such example centrifuge is a Beckman, Allegra X15R centrifuge with swinging bucket rotor SWX750A at 3250 xg. Centrifuging the resulting supernatant results in a 10 fold concentration of greater than 99% of proteins in excess of 300 kDa in the retentate above the membrane. Stated another way, centrifuging the resulting supernatant results a significant depletion of high molecular weight, bulk proteins. Said high molecular weight, bulk proteins increase the viscosity of a solution containing the resulting supernatant and interfere with downstream concentration.

At 112, the filtrate is concentrated to generate concentrated filtrate. Following centrifugation, the filtrate is recovered and the retentate is removed. The first membrane is replaced with a second membrane. One such example second membrane is a Pall insert OD030C65 with a MWCO of 30 kDa. In this example embodiment, the filtrate is concentrated by placing the filtrate is in an upper chamber of the centrifugal filter device having the second membrane and centrifugation is repeated resulting in a concentrated retentate depleted of bulk protein (e.g., proteins over 30 kDa) but having a 10 fold increased concentration in growth factors and cell adhesion promoting proteins as compared to the original resulting supernatant (.e.g., the supernatant recovered at step 108 of the method 100).

At 114, the concentrated filtrate generated is transferred directly into a cell culture vessel to coat a growth surface. In one example embodiment, the concentrated filtrate is added to the cell culture vessel by a peristaltic pump. In one example embodiment, the temperature in the cell culture vessel is between 35° C. to about 40° C. In one example embodiment, the temperature in the cell culture vessel is 37° C.

At 116, the cell pellet (e.g., generated at step 106) is resuspended. In one example embedment, step 116 occurs contemporaneously with step 110. In another example embodiment, step 114 occurs at any time after step 108. In this example embodiment, the cell pellet is combined and resuspended in media (e.g., such as in 20 mL of basal media) to create a resuspended cell pellet. In some example embodiments, a plurality of cell pellets are combined and resuspended.

A hemocytometer and trypan blue (made by Life Technologies) are used for a cell count and a cell viability check. The resuspended cell pellet is centrifuged (e.g., at 400xg for 10 min) resulting in a cell pellet supernatant and a second cell pellet. In this example embodiment, the resulting cell pellet supernatant is aspirated.

At 118, an MSC seeding density is adjusted to a target plating density. In one example embodiment, the target plating density is between 1,200 and 15,000 per cm². Using the cell count calculations from above, the second pellet cells are resuspended at a target plating density in a complete media generating resuspended target cells, the resuspended target cells having the target plate density. In some embodiments, a second cell count is performed to confirm the resuspension at the target plating density.

At 120, the resuspended target cells are added to the cell culture vessel having the coated growing surface (e.g., as coated at step 114). In one example embodiment, the resuspended target cells are added to the cell culture vessel by the peristaltic pump.

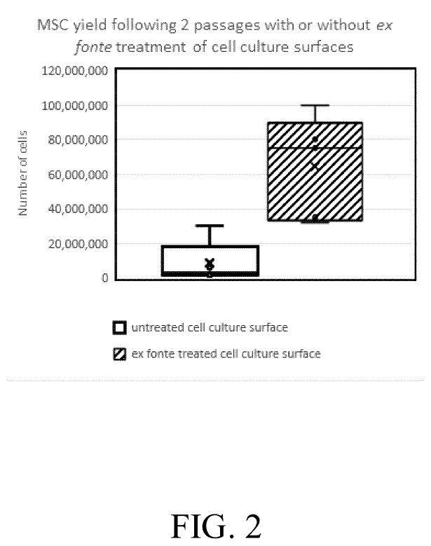

The concentrated filtrate, when applied to a cell culture treated surface prior to plating of the resuspended target cells, results in a ten (10) fold increase in initial adherence of cells from identical suspensions when comparing to untreated cell culture surfaces. As illustrated in FIG. 2, a comparison of ten isolations conducted with and without ex fonte treatment of cell culture surfaces (e.g., as generated by method steps 102-120) reveals that the rate of proliferation was elevated on treated surfaces and the average yield of cells after 2 passages was 8,600,000 cells on untreated surfaces and 64,400,000 cells on treated surfaces. The MSCs undergo an expansion, during which the cell culture undergoes growth. In one example embodiment, the growth of the cell culture is grown for 3-10 days, at 37° C., with 5% carbon dioxide.

At 122, MSCs grown in the cell culture vessel are harvested. At 124, extracellular vesicles (EVs), Exosomes (EXs) and micro vesicles (MVs) grown in the cell culture vessel are harvested. In one example embodiment, step 124 is performed after step 122. In one example embodiment, EX harvest occurs in parallel with or very shortly after passaging of the MSCs. In another example embodiment, harvest of EXs from MSC media occurs without disruption of the MSCs. In yet another example embodiments, the EXs are automatically harvested in parallel using a tangential flow filtration (TFF) while the MSCs are being dissociated and passaged.

The method 100 also increases the number of EVs, EXs and MVs that are harvested, as well as expanding the number of stem cells that are harvested. In addition to the 5-10 billion MSCs that are harvested after expansion from Wharton's jelly, a “second harvest” of EVs comprising mostly EXs are harvestable from the WJ-MSC media after the stem cells are extracted. The EV, EX, and MV secretions from the MSCs are potent. The EV, EX, and MV secretions amount to about 150 trillion EVs, Exs and MVs—predominately and almost entirely EXs. Stated another way, the EV, EX, and MV secretions amount to second harvest of between 15,000-60,000 EX per dose depending on the dosage size.

During the expansion process (e.g., between steps 120 and 122) MSCs produce EVs that are understood be responsible for part of the beneficial effects of MSCs. The coated growth surface generated at step 114 maximizes initial adherence and proliferation of the MSCs and production of EVs throughout the expansion process.

After the harvest at step 122 of the MSCs, stem cell conditioned media (e.g. the media that remains after MSC growth) is drawn from the cell culture vessel for harvesting. In one example embodiment, the stem cell conditioned media is removed from the cell culture vessel by a peristaltic pump with two inline filters. In one example embodiment, the primary filter has a pore size of 0.22 μm to clarify and sterile filter the stem cell conditioned media. In one example embodiment, an outlet of the primary filter is connected to an inlet of a secondary filter. In one example embodiment, the secondary filter is a tangential flow filter that retains molecules larger than 30 kDa. In this example embodiment, the combination of the primary and secondary filters concentrates extracellular vesicles that are between 40 nm to about 400 nm in diameter, while removing larger cell fragments and/or non-adherent cells. Advantageously, the primary and secondary concentrate the EVs, while other elements present in the stem cell conditioned media are not concentrated. Stated another way, unlike concentration by evaporation the concentration of small molecules in the filtrate are the same because salts, amino acids, vitamins and sugars pass through the primary and secondary filters easily while extracellular vesicles do not.

As generated at 124 of method 100, the harvest of EVs (e.g., between 40 nm to about 200 nm in diameter) is concentrate-able from a range of 12.5×10¹² EV's per liter of stem cell conditioned media up to 45×1012 EV per liter. The initial harvest of the stem cell conditioned media (e.g., serum-free, xeno-free media) is typically composed of up to 1.45×10¹² EVs per mL. To provide an example, this system has been used to concentrate the EVs from 5 liters to a volume of less than 0.2 liters allowing for efficient storage of highly concentrated MSC-EV stocks.

The utilization of MSCs for cell therapy applications requires comprehensive testing of said MSCs. In one example embodiment, samples of the MSCs and the media in which they were grown are and tested for potential pathogens including hepatitis, HTLV I/II, CMV & HIV I/II among many others. Additionally, in some example embodiments, sterility endotoxin and mycoplasma testing are performed.

Concentration of EVs in parallel with passaging or harvesting of MSCs has the added benefit of enabling application of MSC safety testing to be performed on the EVs made by those MSCs. Provided that the cells and media for testing are collected at the same time parallel processing begins, all testing for MSC safety is also valid for the extracellular vesicles made by those cells.

Safety and sterility testing of advanced therapeutics is an exceptionally expensive undertaking and use of this system avoids the duplication of those costs that would be required if the processes were to be performed in series rather than in parallel.

MSCs are tools for treating degenerative changes in joints and to reconstruct bones and cartilage. Further, MSCs can be use used in plastic surgeries, aesthetic medicine, cardiovascular diseases, endocrine and nervous system diseases, cell transplantation, and in the repair of damaged musculoskeletal tissues. Investigations of such treatments can be found in Parekkadan B, Milwid J M. Mesenchymal stem cells as therapeutics. Annu Rev Biomed Eng. 2010 Aug. 15;12:87-117. doi: 10.1146/annurev-bioeng-070909-105309. PMID: 20415588; PMCID: PMC3759519, which is incorporated herein by reference in its entirety for all purposes.

EVs, which include exosomes and microvesicles, mediate intercellular communication and play a critical role in nervous system development, function, and repair. EVs can be used as therapeutic agents, focusing on their ability to deliver biomolecules such as proteins, lipids, and nucleic acids to target cells, thereby modulating their function and promoting neuroregeneration. EVs can be used for treatments for a variety of nerve disorders, including neurodegenerative diseases, spinal cord injuries, and peripheral neuropathies. Investigations of such treatments can be found in Galieva L R, James V, Mukhamedshina Y O, Rizvanov A A. Therapeutic Potential of Extracellular Vesicles for the Treatment of Nerve Disorders. Front Neurosci. 2019 Mar. 5;13:163. doi: 10.3389/fnins.2019.00163. PMID: 30890911; PMCID: PMC6411850, which is incorporated herein by reference in its entirety for all purposes.

Exosomes have a wide range of treatment applications in the cosmetics industry. Exosomes deliver active ingredients directly to the skin cells, which is more effective than traditional remedies and will supplement or replace other ingredients used today. Exosomes protect the skin from damage caused by ultraviolet radiation. Exosomes derived from human stem cells were able to promote the growth of new skin cells and improve the appearance of wrinkles. Exosome-based cosmetics have several advantages over traditional cosmetics. First, exosomes target specific cells and deliver their contents directly to those cells, allowing targeted delivery of the active ingredients in the cosmetic. Second, exosomes are non-toxic and do not induce an immune response. This makes exosomes well-suited for use in people with allergies or sensitivities to traditional cosmetics. Finally, exosomes are biodegradable and do not accumulate in the environment.

In one example embodiment, exosomes are generated MSCs, and/or during generation of MSCs, such as the method 100 of FIG. 1. Exosomes are generated through the method 100 of FIG. 1. Exosomes are isolated from MSCs, they can be used as a delivery system for a variety of active ingredients, such as cytokines and growth factors. For example, exosomes deliver anti-aging proteins to the skin, providing a powerful new way to combat wrinkles and other signs of aging. Exosomes have cosmetic applications, such as use of exosomes in cosmeceuticals. Exosomes promote wound healing, protect against UV damage, and increase collagen production. Exosomes reduce inflammation and improve the appearance of fine lines and wrinkles.

In the foregoing specification, specific embodiments have been described. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. In one non-limiting embodiment the terms are defined to be within for example 10%, in another possible embodiment within 5%, in another possible embodiment within 1%, and in another possible embodiment within 0.5%. The term “coupled” as used herein is defined as connected or in contact either temporarily or permanently, although not necessarily directly and not necessarily mechanically. A device or structure that is “configured” in a certain way is configured in at least that way, but may also be configured in ways that are not listed.

To the extent that the materials for any of the foregoing embodiments or components thereof are not specified, it is to be appreciated that suitable materials would be known by one of ordinary skill in the art for the intended purposes.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A method of growing stem cells for therapeutic administration in a subject in need thereof, the method comprising:
separating stem cells and a protein mix from a Wharton's Jelly matrix;
separating the stem cells from the protein mix;
depleting bulk proteins from the protein mix to generate a depleted protein mix;
enriching the depleted protein mix to generate an enriched protein mix; and
combining the stem cells with the enriched protein mix.

2. The method of claim 1, further comprising coating a growth surface with the enriched protein mix prior to the combining the stem cells.

3. The method of claim 1, comprising obtaining the Wharton's Jelly matrix from one or more umbilical cords.

4. The method of claim 1, comprising harvesting the stem cells grown in the enriched protein mix.

5. The method of claim 1, comprising harvesting the extra cellular vesicles (EVs) grown in the enriched protein mix.

6. The method of claim 5, filtering EVs through a primary and secondary filter, wherein the output of the primary filter is the inlet of the secondary filter.

7. The method of claim 6, wherein the primary filter has a pore size of 0.22 μm and the secondary filter retains molecules larger than 30 kDa.

8. A MSC generated by the method of claim 1.

9. An extra cellular vesicle (EV) generated by the method of claim 1.

10. A method of treating a cancer, nerve disorders, or an infectious disease in a subject comprising administering to the subject a MSC generated by the method of claim 1.

11. A method of treating autoimmune disease, or an infectious disease in a subject comprising administering to the subject an EV generated by the method of claim 1.

12. A method of deriving extracellular vesicles (EVs) from stem cells for administration in a subject in need thereof, the method comprising:
separating stem cells and a protein mix from a Wharton's Jelly matrix;
separating the stem cells from the protein mix;
depleting bulk proteins from the protein mix to generate a depleted protein mix;
enriching the depleted protein mix to generate an enriched protein mix;
combining the stem cells with the enriched protein mix;
incubating the stem cells with the enriched protein mix to generate stem cell enriched media; and
harvesting the extracellular vesicles generated therefrom.

13. The method of claim 12, further comprising removing the stem cells from the enriched protein mix, prior to the harvesting.

14. The method of claim 12, the administration comprising topical administration to a skin surface of the subject.

15. The method of claim 13, comprising performing a single safety test on the stem cells and stem cell enriched media to determine the safety status of the stem cells and the EVs.

16. An EV generated by the method of claim 12.

17. A method of treating autoimmune disease, or an infectious disease in a subject comprising administering to the subject an EV generated by the method of claim 12.

18. The method of claim 12, comprising wherein the stem cell enriched media, the protein mix, the stem cells, and the EVs are xeno-free and serum free.

19. A method for preparing a population of mesenchymal stem cells (MSC) for therapeutic administration to a subject in need thereof, the method comprising:
(a) separating stem cells from a protein mix; (b) depleting bulk proteins from the protein mix to generate a depleted protein mix; (c) enriching the depleted protein mix to generate an enriched protein mix; (d) concentrating stem cells; (e) combining the stem cells with the enriched protein mix; (f) incubating the stem cells with the enriched protein mix to generate stem cell enriched media; and (g) harvesting MSCs generated therefrom.

20. The method of claim 19, comprising wherein the stem cell enriched media, the protein mix, and the stem cells are xeno-free and serum free.
